(19) United States
(12) Patent Application Publication
(10) Pub. No.: US 2007/0027382 A1
(43) Pub. Date: Feb. 1, 2007

(54) SYSTEM FOR MONITORING OF PATIENTS

(76) Inventors: Herbert L. BERMAN, Los Altos Hills, CA (US); Robert N. BLAIR, San Jose, CA (US); James W. MOYER, San Francisco, CA (US); Russell O. POTTS, San Francisco, CA (US)

Correspondence Address: LEVINE BAGADE HAN LLP, 2483 EAST BAYSHORE ROAD, SUITE 100, PALO ALTO, CA, 94303, US

(21) Appl. No.: 11/422,561

(22) Filed: Jun. 6, 2006

Related U.S. Application Data
(63) Continuation-in-part of application No. 10/632,991, filed on Aug. 1, 2003.
Application No. 10/632,991 is a continuation-in-part of application No. 09/844,687, filed on Apr. 27, 2001, now Pat. No. 6,748,250.

(51) Int. Cl. A61B 5/05; A61B 5/00

(52) U.S. Cl. 600/347; 600/316; 600/365

(57) ABSTRACT

A patient monitoring system implemented by a service provider for users via recording a patient's analytes measurements by non-invasively interrogating a skin patch placed upon the patient's skin surface. The system includes an input module to provide a device to measure analytes from a patient, such as a measurement of the blood glucose level. The measurement is shared among a plurality of output devices such as computers, personal digital assistants (PDAs), cellular phones, and pagers that are stationed or held by various users, such as doctors, patients, researchers, pharmacies, labs, and health insurers. In addition, behavioral attributes are recorded and correlated with the analytes measurements to generate a profile. The profile is selectively sent to output devices based on the user profile corresponding to the output device. Also, access to the profile is monitored by a security module that encrypts the profile to prevent access by un-authorized users.

### CROSS-REFERENCE TO RELATED APPLICATIONS

This is a continuation-in-part of U.S. patent application Ser. No. 10/632,991 filed Aug. 1, 2003, which is a continuation of U.S. patent application Ser. No. 09/844,687 filed Apr. 27, 2001 (now U.S. Pat. No. 6,748,250 B1), each of which is incorporated herein by reference in its entirety.

### FIELD OF THE INVENTION

The present invention relates generally to devices and methods for improving the delivery of patient information and care to patients, particularly to transactions involved in utilizing a non-invasive monitoring system to deliver physiological information to patients and patients' service providers.

### BACKGROUND OF THE INVENTION

Numerous diseases require the monitoring of various physiological attributes of a patient. These attributes such as blood glucose level and other blood analyte levels are invaluable to patients and health service providers such as doctors, medical professions, pharmacies, researchers, insurance companies, and government agencies.

Particularly in patients with diabetes, monitoring the level of blood glucose is extremely important in controlling the patient's health, and decreasing or delaying the damaging effects of uncontrolled blood glucose. Diabetes is a disease in which the body does not produce or properly use insulin, which results in the increase uptake of glucose from the blood across cell membranes. About sixteen million people in the United States are diabetics. The American Diabetes Association reports that diabetes is the seventh leading cause of death in the United States. The complications of the disease include blindness, kidney disease, nerve disease, heart disease, and death.

Specifically, for diabetes, monitoring various physiological attributes is essential for diabetic patients. For example, it is essential that patients practice frequent self-monitoring of blood glucose (SMBG). Based upon the level of glucose in the blood, individuals may make insulin dosage decisions before injection. Monitoring the trends in blood glucose over time provides health care providers with invaluable information on the adequacy of therapy, the compliance of the patient and the progression of the disease. However, the prior systems of glucose monitoring usually required obtaining blood from a finger stick (invasive method) or obtaining body fluids (other than blood) and subcutaneous tissue (also an invasive method).

Examples of non-invasive glucose monitoring system, as illustrated in U.S. Pat. No. 6,424,851 (Berman et al.) and in U.S. Pat. Pub. 2006/0004271 A1 (Peyser et al.), each owned by the assignee of this application and incorporated herein by reference in its entirety, provide solutions for non-invasively gathering blood glucose information from diabetic patients. Use of a non-invasive technology rather than an invasive technology permits a significantly better approximation to continuous monitoring, which in turn may contribute significantly to improved health care for diabetic patients. Other examples of optically-based patient monitoring systems which utilize non-invasive glucose-measuring devices are also illustrated in U.S. Pat. No. 6,748,250 (Berman et al.) and in U.S. Pat. Pub. 2004/0097796 A1 (Berman et al.), each of which is also owned by the assignee and is incorporated herein by reference in its entirety.

Therefore, it is advantageous to have a monitoring system that leverages on other non-invasive glucose-measuring devices to provide a medium for sharing of the monitored information.

### BRIEF SUMMARY OF THE INVENTION

The patient monitoring system in accordance with one variation may have one or more non-invasive analyte monitor devices, a data processing and storage unit, and one or more information recipients. All elements of the system can be linked to communicate with each other via a network or wireless protocol.

The data processing and storage unit may implement for a user a monitoring system that organizes and processes physiological and behavior attributes of the user to enable transmission of these attributes to information recipients. Optionally, the data processing and storage unit can be programmed to send automated warnings such as by email, phone, or fax to a patient or information recipients if the patient's condition falls outside an acceptable limit that can be prescribed by the patient's caregiver or physician.

Generally, a patient monitoring system for distributing information among one or more recipients may typically comprise an analyte monitoring device configured to measure at least one attribute of a patient via non-invasively interrogating a skin patch placed upon a skin surface of the patient, and a data processing unit to process the at least one attributes and to generate and transmit a profile of the patient to one or more recipients.

One method of monitoring a patient may generally comprise measuring a physiological attribute of the patient from a patch placed upon a skin surface of the patient, and transmitting the attribute to one or more users. More particularly, other methods may generally comprise interrogating the skin patch placed upon the skin surface of the patient via a non-invasive measurement device, correlating a measurement from the skin patch to a physiological attribute of the patient, and transmitting the attribute to one or more users.

### BRIEF DESCRIPTION OF THE SEVERAL VIEWS OF THE DRAWINGS

FIG. 1 is a block diagram of an example of a patient monitoring system.

FIG. 2 illustrates a more detail illustration of the internal architecture of a non-invasive monitor device 210, a processing unit and storage system 510, and information recipients 600.

FIG. 3 illustrates a flow chart of registering information into the monitor device.

FIG. 4 illustrates an example of the step-by-step information flow from the monitor device to a data processing and storage system.

FIG. 5 is a flow chart of the information processing in a data processing unit.

### DETAILED DESCRIPTION OF THE INVENTION

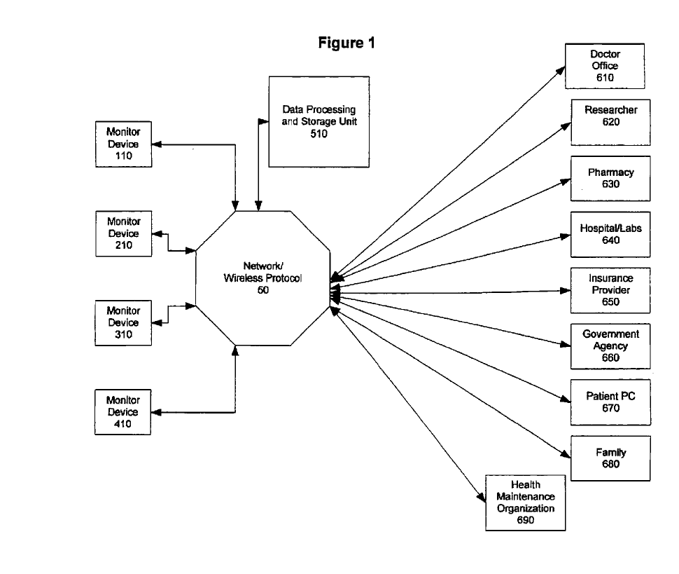

Referring to FIG. 1, the elements of the patient monitoring system in accordance with one variation may have one or more non-invasive analyte monitor devices 110, 210, 310, 410, a data processing and storage unit 510, and one or more information recipients 600, which may include, but is not limited to, exemplary recipients such as doctor's office 610, researcher 620, pharmacy 630, hospital/labs 640, insurance provider 650, government agency 660, patient 670, family member 680, and/or health maintenance organization 690, etc. All elements of the system can be linked to communicate with each other via a network or wireless protocol 50.

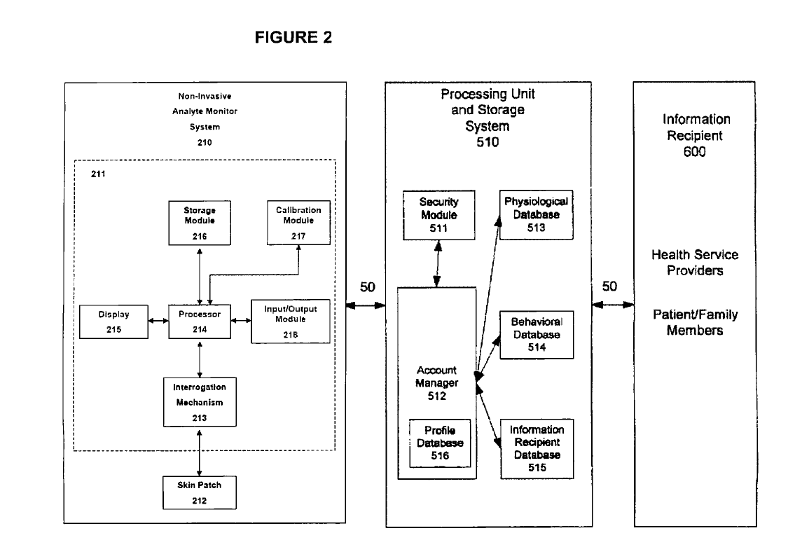

As illustrated in more detail in FIG. 2, the data processing and storage unit 510 may implement for a user a monitoring system that organizes and processes physiological and behavior attributes of the user to enable transmission of these attributes to information recipients 600. Optionally, the data processing and storage unit 510 can be programmed to send automated warnings such as by email, phone, or fax to a patient or information recipients if the patient's condition falls outside an acceptable limit that can be prescribed by the patient's caregiver or physician.

In one example of the system where the user is a diabetic patient, the user may utilize a non-invasive analyte monitoring system 210 based on methods and devices 211 configured to measure glucose from skin patches 212 which collect and retain the glucose brought to the skin surface. Examples of non-invasive analyte monitoring systems 210 which utilizes glucose obtained from a skin patch 212 may be seen in further detail in U.S. Pat. Pub. 2006/0004271 A1 (Peyser et al.), which has been incorporated by reference above. Generally, the patch 212 may be placed on any suitable skin surface such as a finger, palm, wrist, forearm, etc. Such a patch 212 may generally have a collection layer, a detector, and an adhesive layer for adhering the patch 212 to the user's skin surface. The detector may generally comprise any number of detectors which are capable of detecting nanogram quantities of glucose, such as a dry, polymer-based electrochemical sensor, a wet electroenzymatic sensor in a microfluidic package, a glucose-sensitive fluorescent molecule or polymer, etc. The collection layer may generally comprise a fixed volume reservoir to help minimize the effects of a user's sweat rate.

The measurement device 211 may generally comprise a sensitive measurement mechanism for interrogating and measuring the glucose from the patch 212 and converting this measurement into a glucose concentration. The device 211 may generally include an interrogation mechanism 213 which is used to interrogate and detect the collected glucose from the patch 212. The type of interrogation mechanism 213 may depend upon the type of patch 212 utilized; for instance, if the patch 212 were configured as an electrochemical detector, the interrogation mechanism 213 may be correspondingly configured as an electrochemical sensor.

Generally, prior to application of the patch 212 to the user's skin surface, the skin may be wiped clean to remove any residual glucose remaining on the skin. The wipe may include any number of supports capable of absorbing a solvent or having a solvent impregnated therein, for example, any type of fabric, woven, non-woven, cloth, pad, polymeric, or fibrous mixture, etc. The solvent absorbed in the wipe typically does not contain solvents, markers, or other chemicals that would interfere with the measurement of glucose. Polar solvents, for example, a mixture of distilled water and alcohol, may be utilized.

In some variations, the wipe may also contain a marker that is deposited upon the skin prior to patch placement. The marker may comprise a chemical having a short half-life so as to decay after a short period of time; alternatively, the marker may also be bound to a volatile compound made to evaporate in a short period of time. Such a marker may be deposited onto the skin by the wipe so as be detectable by the device 211. If the marker is detected by the device 211, then the measurement may proceed; however, if the marker is not detected, the measurement does not proceed. In this way, the user can have some indication that the skin has not been properly wiped and any possibly erroneous readings may be prevented.

The device 211 may also include a processor 214 for analyzing the measured data and processing the information for display to the user via a graphical display 215, which may also be utilized to display a variety of other information. The device 211 may also include a computer-executable code containing a calibration module 217, which relates measured values of the detected glucose to blood glucose values. Furthermore, a storage module 216 in device 211 may be utilized for storing measurements and user-related information, which may be inputted via a number of input/output modules 218, such as buttons and other types of user interface mechanisms.

Alternatively and/or additionally, the user may also input behavioral attributes such as time duration between analyte measurement and last meal, time duration between analyte measurement and last exercise session, time duration between analyte measurement and last resting session, time and dosage of medication taken, etc., via the input/output module 218. These behavioral attributes may affect the interpretation of the blood glucose measurement. For example, blood glucose level tends to be higher for users that have just eaten a meal. Thus, by adding behavioral attributes, the system 210 can provide a better profile of the user's health to information recipients 600. Also, the user may utilize the input/output module 218 to include other physiological attributes such as heart rate, blood pressure, etc. Optionally, the input/output module 218 can comprise an activity sensor that determines energy use and/or a metabolic activity sensor that measures metabolic rates such as oxygen consumption.

Additionally, the output module 218 of the analyte system 210 may serve as a messaging terminal for the patient. These messages can be configured as automatic alarms that alert the patient when the analyte measurements, behavior attributes, physiological attributes, etc., are out of a normal range prescribed by the patient, the patient's caregiver, and/or the patient's physician. These messages can be generated by the analyte device 211 itself or from any one of the information recipients. For example, if the patient's physician determines that the patient is not responding to a prescribed medicine dosage, the physician can send a message to the display 215 via the input/output module 218 to request the patient to change his dosage or to request a visit to the physician's office for consultation.

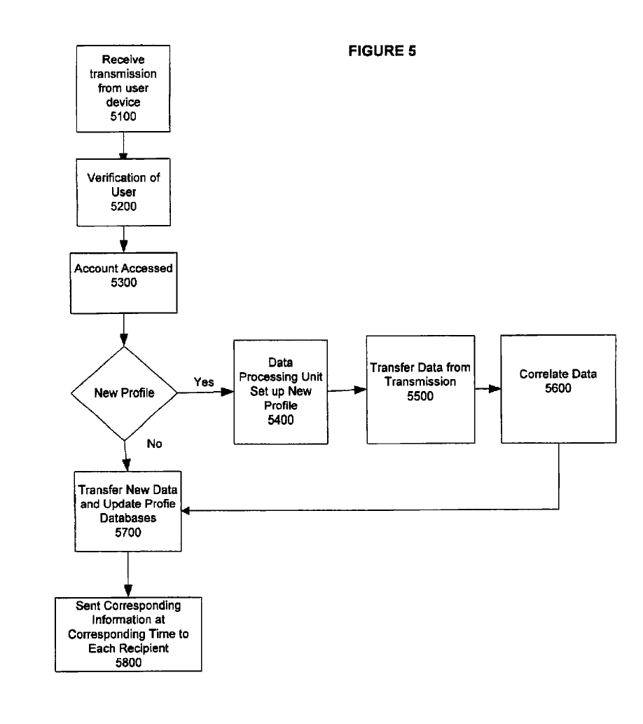

All the analyte measurements, behavior attributes, and physiological attributes are communicated to the data processing and storage unit 510 for processing and storage, which will be furthered described in detail in FIG. 5. These attributes are sent to the data processing and storage unit 510 via a network such as the Internet, local area network (LAN) and/or wide area network (WAN), wireless and/or wired, or other network infrastructure 50. In one variation, the monitor device 211 has its own wireless transmission module. In an alternative variation of the wireless transmission, the monitor device 211 is coupled to a wireless device such as a cellular phone, a pager or a wireless modem to enable transmission. Optionally, due to the large amount of data being collected, the monitor device 211 may send all information to a local terminal and storage located within a patient's home, a physician's office, or a hospital. The information to the local terminal can be transmitted over a short-range radio frequency (RF) link (e.g. Blue tooth). Subsequently, the information stored at the local terminal will be communicated to the processing unit 510.

After communication is established between the monitor device 211 and the processing unit 510, an account manager 512 in the processing unit 510 accesses the user's account and the security module 511 verifies the user's identity via a password or any other security means. After verification, the attributes are transmitted and organized into a physiological database 513, which stores the user's analyte measurements and other physiological attributes, and a behavioral database, which stores the user's behavioral attributes. The account manager 512 also communicates with an information recipient database 515 that includes the user's selected information recipients 600 and recipient parameters associated with each specific information recipient. These “recipient parameters” as described herein are requirements that direct the transmission of the user attributes, which may include “type of information” such as report of blood glucose level, an email to alert if blood glucose level reaches a certain maximum or minimum, a report of behavioral and blood glucose correlation, “time of information” such as weekly, monthly, or quarterly, “fonnat type” such as a graphical representation or text, and “information recipient” such as sending the information to doctor and patient personal computer, or sending to family members in case of emergency.

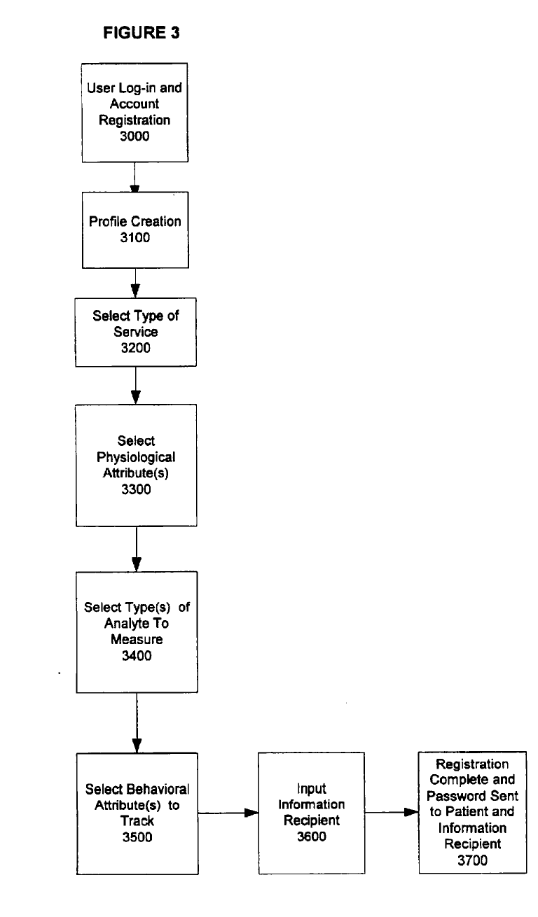

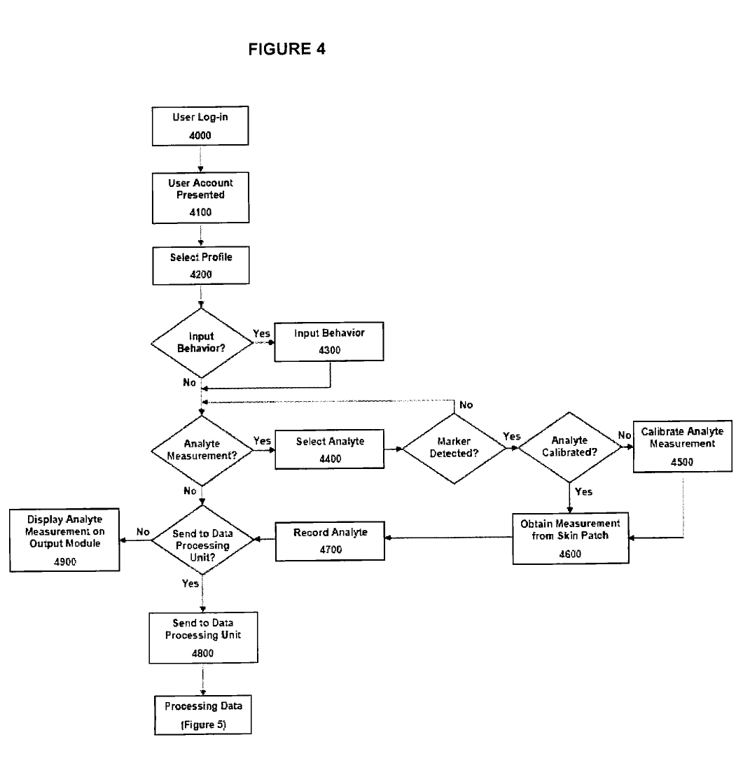

The processes of gathering the user's attributes, processing the attributes, and transmitting the attributes to corresponding information recipients are furthered described in detail in FIG. 3, which illustrates the registration of the user, in FIG. 4, which illustrates the gathering of the user's attributes, and in FIG. 5, which illustrates the data processing and transmission of the attributes.

FIG. 3 illustrates the events that take place in user registration. The user can register via the monitor device 211 or any computing machine that enables communication to the processing unit 510. The user logs-in and account registration is initiated 3000 if user has not registered. The user provides account information (e.g. name, address, date of birth, prior medical history, or monitor device serial number). The user then creates 3100 a profile, which is a set of data relating to a specific service (e.g. monitoring the blood glucose level or monitoring alcohol level) by selecting 3200 the type of service needed such as analyte measurement reading, analysis and tracking of physiological and behavioral attributes, transmitting information among information recipients, or any combination of the above mentioned services. After service is selected 3200, the user selects one or more physiological attributes to track 3300, one or more analytes to be measured 3400, and one or more behavioral attributes to track 3500. If information transmission among recipients is selected in step 3200, the user needs to input all recipients' information and recipient parameters 3600. The profile may be stored in the storage module 216 of the monitor device 211 and in a profile database 516 in the account manger 512 of the processing unit 510. Alternatively, the profile can be stored on either the storage module 216 or the account manger 512. Profile is completed 3700 and the data processing unit sends a confirmation with password to the user and his list of recipients. Alternatively, the user can create numerous profiles within the same account.

After user registration, the user can utilize his device to gather physiological and behavioral attributes, which is illustrated in FIG. 4. The user logs in 4000 to the device 211 with password from the registration and user account is presented by the input module 4100. Altermatively, user log-in can be accomplished by voice recognition or by a fingerprint. The user selects the desired profile 4200. If behavior inputs are required, the user inputs 4300 the behavior attributes such as “time duration between analyte measurement and last meal”, “time duration between analyte measurement and last exercise session”, “time duration between analyte measurement and last resting session”, or “whether other drugs or alcohol was taken prior to measurement.” If no behavioral attributes are required, the user is presented with opportunity to select analyte measurement. If selected, the user selects the desired analyte to be measured 4400 and if calibration is needed, the calibrator module is initiated to calibrate 4500 the processor 214.

In variations where a marker is deposited upon the skin surface by a wipe, once the user has selected the desired analyte to be measured 4400, the device 211 may initially interrogate the skin patch 212 to detect the presence of a marker. If the marker is detected, then the analyte calibration may be initiated, if necessary; otherwise, of the marker is not detected, thus indicating an improperly wiped skin surface, then the measured is prevented from proceeding until such a marker is detected by the device 211.

If calibration is complete, the user can obtain measurements from the skin patch 4600 and the analyte measurement generator records 4700 and calculates the analyte level. If only the analyte measurement is selected and no processing is needed, the display 215 will display the measurement. If further processing is required, the data is sent 4800 to the processing unit and the date is processed, as illustrated in more detail in FIG. 5.

In FIG. 5, the data processing unit 510 receives transmission from the monitor device 5100. The user is verified 5200 via the security module and user account is accessed 5300 by the account manager. If the transmission pertains to an existing profile, the data is transferred and the profile database in the account manager is updated 5700. If the transmission consists of data pertaining to a new profile, a new profile is created by the processing unit 5400 and data is transferred 5500. The processing unit then organizes and correlates the data according to the behavioral and physiological relationships and recipient parameters 5600 and updates the profile database in the account manager 5700. After updating the profile database 5700, the account manager is responsible for sending out the corresponding reports and profiles at the corresponding time to each recipient based on the recipient parameters 5800. Alternatively, the reports and profiles are encrypted and access is only granted to recipients with valid passwords to prevent unauthorized use.

Foregoing described embodiments of the invention are provided as illustrations and descriptions. They are not intended to limit the invention to precise form described. In particular, it is contemplated that functional implementation of invention described herein may be implemented equivalently in hardware, software, firmware, and/or other available functional components or building blocks. Other variations and embodiments are possible in light of above teachings, and it is thus intended that the scope of invention not be limited by this Detailed Description, but rather by the claims following.

We claim:

1. A method of monitoring a patient, comprising:
measuring a physiological attribute of the patient from a patch placed upon a skin surface of the patient; and
transmitting the attribute to one or more users.

2. The method of claim 1 wherein measuring comprises measuring a blood glucose level of the patient.

3. The method of claim 1 wherein measuring comprises measuring the attribute via an electrochemical sensor.

4. The method of claim 1 further comprising processing one or more attributes to generate a profile of the patient.

5. The method of claim 1 further comprising correlating the attribute with one or more behavioral attributes in generating the profile of the patient.

6. The method of claim 5 further comprising encrypting the profile to prevent unauthorized access.

7. The method of claim 1 wherein transmitting comprises transmitting the attribute wirelessly.

8. The method of claim 7 wherein transmitting wirelessly comprises transmitting the attribute via a cellular phone.

9. The method of claim 7 wherein transmitting wirelessly comprises transmitting the attribute via a wireless transmitting device.

10. The method of claim 1 wherein transmitting comprises transmitting the attribute via a network.

11. A method of monitoring a patient, comprising:
interrogating a skin patch placed upon a skin surface of the patient via a non-invasive measurement device;
correlating a measurement from the skin patch to a physiological attribute of the patient; and
transmitting the attribute to one or more users.

12. The method of claim 11 wherein interrogating comprises electrochemically interrogating the skin patch.

13. The method of claim 11 wherein interrogating comprises measuring a blood glucose level of the patient.

14. The method of claim 11 wherein correlating comprises processing the measurement to generate a profile of the patient.

15. The method of claim 14 further comprising encrypting the profile to prevent unauthorized access.

16. The method of claim 11 wherein transmitting comprises transmitting the attribute wirelessly.

17. The method of claim 16 wherein transmitting wirelessly comprises transmitting the attribute via a cellular phone.

18. The method of claim 11 wherein transmitting comprises transmitting the attribute via a network.

19. The method of claim 11 further comprising determining whether to proceed with the measurement by looking for the presence of a marker upon the skin surface prior to interrogating.

20. A patient monitoring system for distributing information among one or more recipients, comprising:
an analyte monitoring device configured to measure at least one attribute of a patient via non-invasively interrogating a skin patch placed upon a skin surface of the patient; and
a data processing unit to process the at least one attributes and to generate and transmit a profile of the patient to one or more recipients.

21. The system of claim 20 wherein the profile is transmitted via a network.

22. The system of claim 20 wherein the profile is transmitted wirelessly.

23. The system of claim 20 wherein the profile is transmitted based on recipient parameters.

24. The system of claim 20 further comprising a security module to verify recipient access to the profile.
